(19) United States
(12) Patent Application Publication
(10) Pub. No.: US 2025/0288540 A1
(43) Pub. Date: Sep. 18, 2025

(54) METHODS AND COMPOSITIONS FOR PAIN MANAGEMENT

(71) Applicant: SILO PHARMA, INC., ENGLEWOOD CLIFFS, NJ (US)

(72) Inventor: Eric Weisblum, Englewood Cliffs, NJ (US)

(21) Appl. No.: 19/076,967

(22) Filed: Mar. 11, 2025

Related U.S. Application Data
(60) Provisional application No. 63/564,833, filed on Mar. 13, 2024.

(51) Int. Cl. A61K 31/135; A61K 9/00; A61K 31/165; A61K 31/195; A61K 31/381; A61K 31/485; A61K 45/06; A61K 47/34

(52) U.S. Cl. CPC A61K 31/135; A61K 9/0024; A61K 31/165; A61K 31/195; A61K 31/381; A61K 31/485; A61K 45/06; A61K 47/34

(57) ABSTRACT

Disclosed herein include methods, compositions, and kits suitable for use in treating fibromyalgia. In some embodiments, the method comprises the administration of a composition comprising ketamine, norketamine and a pharmaceutically acceptable salt thereof. In some embodiments, the composition is an extended-release formulation, such as an implant.

### RELATED APPLICATIONS

This application claims the benefit under 35 U.S.C. § 119 (e) of U.S. Provisional Patent Application Ser. No. 63/564,833, filed Mar. 13, 2024, the content of this related application is incorporated herein by reference in its entirety for all purposes.

### BACKGROUND

### Field

The present disclosure relates generally to the field of methods, compositions, and kits suitable for use in treating pain, such as fibromyalgia. In particular, the method comprises the administration of a composition comprising ketamine, norketamine and a pharmaceutically acceptable salt thereof, which is an extended-release formulation, such as an implant.

### Description of the Related Art

Fibromyalgia is a chronic disease involving widespread pain, among other symptoms. Fibromyalgia occurs in approximately 2% of the United States general population with a higher incidence in women (3.4%) compared to men (0.5%). While the etiology of fibromyalgia is unknown, there is evidence to suggest some abnormalities in central monoaminergic neural transmission including serotonin and norepinephrine systems. Tricyclic antidepressants (TCAs) and serotonin-norepinephrine reuptake inhibitors (SNRIs) have been explored as a treatment for fibromyalgia. TCAs including amitriptyline, and cyclobezaprine showed modest efficacy with improvements in self-ratings of pain, stiffness, fatigue, sleep and tenderness. However, antidepressants used to treat fibromyalgia show adverse reactions in a significant percentage of patients, such as increased tolerability after the progressive introduction of antidepressants, mouth dryness, constipation, urinary and bowel emptying difficulties, sedation, drowsiness and orthostatic hypotension. Limited and inconsistent results were also obtained for SRIs including fluoxetine and citalopram. Common side effects of SRI are associated with the actions of serotonin and include nausea, gastric discomfort, vomiting, anorexia, diarrhea and skin hyperhydrosis. Antiepileptic drugs, including gabapentin and pregabalin, have been prescribed to patients with neuropathic pain subtypes and fibromyalgia. Pregabalin was the first drug approved for fibromyalgia in 2007 by the FDA. Pregabalin has demonstrated efficacy in several recent fibromyalgia trials with improved pain scores, quality of sleep and fatigue. However, common side effects of pregabalin include dizziness, sleepiness and weight gain. Thus, while the existing drugs showed reduced pain in some patients, there is still room for improvement in both efficacy and side effect profiles.

### SUMMARY

Disclosed herein include methods for treating fibromyalgia in a subject in need thereof. In some embodiments, the method comprises administering to the subject an implant comprising (i) a composition comprising at least one selected from the group consisting of ketamine, norketamine and a pharmaceutically acceptable salt thereof, and (ii) a polymer.

In some embodiments, the implant comprises enantiomerically pure S-(+)-ketamine, enantiomerically pure R-(−)-ketamine, or a racemic mixture of S-(+)-ketamine and R-(−)-ketamine. In some embodiments, the implant comprises ketamine hydrochloride.

In some embodiments, polymer is acid-terminated or ester-terminated. In some embodiments, the polymer is selected from the group consisting of poly (lactic-co-glycolic acid) (PLGA) copolymer, polyesteramide, polyanhydride, polyacetal, polycaprolactone, polycarbonate, and any combination thereof. In some embodiments, the polymer is PLGA copolymer. In some embodiments, the PLGA copolymer has a co-monomer ratio for lactide to glycolide (L/G) content of about 50:50 to about 85:15. In some embodiments, the PLGA copolymer has a co-monomer ratio for lactide to glycolide (L/G) content of 50:50 or 75:25.

In some embodiments, the composition is present in an amount of 10%-50% of the mass of the implant. In some embodiments, the polymer is present in an amount of 50%-90% of the mass of the implant. In some embodiments, the implant is rod-shaped. In some embodiments, the implant has a length between 5-50 mm. In some embodiments, the implant has a length of 20 mm. In some embodiments, the implant has a diameter between 0.1-5 mm. In some embodiments, the implant has a diameter of 1.2 mm. In some embodiments, the implant has a mass of about 0.01-1 g. In some embodiments, the implant has a mass of 0.2-0.8 g.

In some embodiments, the implant is biodegradable. In some embodiments, the implant exhibits a reduced pH internal environment. In some embodiments, the reduced pH internal environment facilitates the release of the composition with enhanced solubility at reduced pH. In some embodiments, the composition is released from the polymer over an extended period of time. In some embodiments, the extended period of time is at least 14 days. In some embodiments, the extended period of time is at least 30 days.

In some embodiments, the subject has a reduced pain intensity score, decreased tenderness, increased muscle endurance, decreased pain threshold and/or decreased pain tolerance. In some embodiments, the pain intensity score is reduced by at least 20%, wherein the pain intensity score is measured by visual analog scale (VAS). In some embodiments, the pain intensity score is reduced by at least 50%, wherein the pain intensity score is measured by visual analog scale (VAS). In some embodiments, the tenderness, pain threshold and/or pain tolerance is decreased by at least 20%. In some embodiments, the muscle endurance is increased by at least 20%.

The method can further comprise concurrently administering to the subject a muscle relaxant. In some embodiments, the muscle relaxant is selected from the group consisting of afloqualone, baclofen, carisoprodol, chlormezanone, chlorphenesin carbamate, chlorzoxasozone, cyclobenzaprine, clonazepam, dantrolene, diazepam, eperisone, idrocilamide, inaperisone, mephenesin, mephenoxalone, methocarbamol, metaxalone, mivacurium chloride, orphenadrine, phenprobamate, pridinol mesylate, quinine, tetrazepam, thiocolchicoside, tizanidine, tolperisone, and pharmaceutically acceptable salts thereof. The method can further comprise concurrently administering to the subject an anti-inflammatory agent. The method can further comprise concurrently administering to the subject an antidepressant. In some embodiments, the antidepressant is duloxetine or milnacipran. The method can further comprise concurrently administering to the subject an additional therapeutic agent selected from the group consisting of dextromethorphan, and pregabalin.

### BRIEF DESCRIPTION OF THE DRAWINGS

FIG. 1 depicts non-limiting exemplary embodiments and data related to the appearance of a DR-0637 batch extrudate.

FIG. 2 depicts non-limiting exemplary embodiments and data related to the appearance of a DR-0638 batch extrudate.

FIG. 3 depicts non-limiting exemplary embodiments and data related to the appearance of a DR-0639 batch extrudate.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for reducing pain in a subject in need thereof. In some embodiments, the method comprises administering to the subject a composition comprising at least one selected from the group consisting of ketamine, norketamine and a pharmaceutically acceptable salt thereof. The composition can comprise ketamine hydrochloride formulated into an implant with a polymer. The composition can be released from the polymer, over an extended period of time.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2^{nd} ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, “chronic pain” refers to a person suffering from persistent, non-acute, sometimes disabling pain in the extremities or other areas of the body, often of unknown origin.

As used herein, the term “ketamine” includes ketamine in its racemic (R/S) form, in its R-(−) enantiomerically pure form, or in its S-(+) enantiomerically pure form.

As used herein, the term “norketamine” includes norketamine in its racemic (R/S) form, in its R-(−) enantiomerically pure form, or in its S-(+) enantiomerically pure form.

As used herein, “enantiomerically pure” refers to compositions consisting substantially of a single isomer (i.e., substantially free of the opposite isomer), preferably consisting of 90%, 92%, 95%, 98%, 99%, or 100% (w/w) of a single isomer. For example, when the methods of the invention include the administration of enantiomerically pure R-(−)-ketamine, the pharmaceutical composition administered can include at least 95% (w/w) S-(+)-ketamine, and less than 5% (w/w) R-(−)-ketamine.

As used herein, “isomerically pure” refers to compositions consisting substantially of a single diastereomer (i.e., substantially free of other isomers), preferably consisting of 90%, 92%, 95%, 98%, 99%, or 100% (w/w) of a single isomer.

As used herein, “abuse” refers to the intentional, non-therapeutic use of a drug, even once, for its psychological or physiological effects.

As used herein, “misuse” refers to the intentional use, for therapeutic purposes, of a drug by an individual in a way other than prescribed by a health care provider or for whom it was not prescribed.

As used herein, “tolerance” refers to a physiological state characterized by a reduced response to a drug after repeated administration (e.g., a higher dose of a drug is required to produce the same effect that was once obtained at a lower dose).

As used herein, a “patient” refers to a subject that is being treated by a medical professional, such as a Medical Doctor (i.e., Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place. In some embodiments, the patient is a human or an animal. In some embodiments, the patient is a mammal. As used herein, the terms “individual,” “host,” “subject,” and “patient” are used interchangeably.

As used herein, “administration” or “administering” refers to a method of giving a dosage of a pharmaceutically active ingredient to a subject.

As used herein, a “dosage” can refer to the amount of active ingredients (e.g., ketamine).

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “emulsion” refers to a liquid discrete phase homogeneously dispersed in a liquid continuous phase.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate the administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well-known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20^{th} ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “dosage regime” refers to drug administration regarding formulation, route of administration, drug dose, dosing interval and treatment duration.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire). “Pre-treatment” or “baseline,” as used herein, refers to the status of a subject prior to administration of a particular therapy.

As used herein, the term “implant” refers to a composition which can be inserted into the subject, e.g., subcutaneously, intramuscularly, etc. In some embodiments, the implant is also removable.

As used herein, the term “long term” refers to a period of time greater than about one month, greater than about two months, greater than about three months, greater than about four months, greater than about five months, greater than about six months, greater than about seven months, greater than about eight months, greater than about nine months, greater than about ten months, greater than about eleven months, greater than about one year or longer.

As used herein, the term “long term delivery system,” “extended-release formulation,” “extended-release system,” and “controlled release formulation” are used interchangeably. They refer to systems which, once administered to the subject, gradually deliver the target therapeutic drug to the subject in an effective amount to treat a disorder. The drug can be delivered, in some embodiments, over a period of greater than about two weeks, greater than about one month, greater than about two months, greater than about three months, greater than about four months, greater than about five months, greater than about six months, greater than about seven months, greater than about eight months, greater than about nine months, greater than about ten months, greater than about eleven months, greater than about one year or longer.

As used herein, the term “enhanced solubility” refers to an increase of at least 10% over solubility at neutral pH. In some embodiments, the increase is at least 20%, at least 30%, at least 40%, at least 50%, at least 60%, at least 70%, at least 80%, at least 100% (2-fold), at least 3-fold, at least 4-fold, at least 5-fold, at least 6-fold, at least 7-fold, at least 8-fold, at least 9-fold, at least 10-fold, at least 15-fold, at least 20-fold, at least 30-fold, at least 40-fold, at least 50-fold, at least 70-fold, at least 100-fold, at least 150-fold, at least 200-fold, at least 300-fold, at least 500-fold, at least 1000-fold or least more than 1000-fold. In some embodiments, the drug exhibits negligible solubility at neutral pH.

As used herein, the term “reduced pH environment” refers to a pH of below 7.0. In some embodiments, the term refers to a pH of below 6.5, below 6.0, below 5.5, below 5.0, below 4.5, below 4.0, below 3.5, below 3.0, below 2.5, or below 2.0. In some embodiments, the term refers to a pH of 5.0, 4.5, 4.0, 3.5, 3.0, 2.5, or 2.0.

As used herein, the term “biodegradable” or “bioresorbable” refers to a material that is degraded in a biological environment and/or inside a living organism. In some embodiments, “biodegradable” refers to a material that has a finite or measurable half-life in a biological environment and/or inside a living organism.

As used herein, the term “polymer” refers to a macromolecule composed of individual units, or monomers. The polymer can be a branched polymer, a linear polymer, a cross-linked polymer or any other type of polymer known in the art.

As used herein, the term “drug load” refers to the amount of drug in the implant as a percentage by mass or by weight. If other materials are present in the implant besides the therapeutic drug and the polymer, the drug load is calculated without considering the other materials.

As used herein, the term “inherent viscosity” or “intrinsic viscosity” are used interchangeably. They refer to a measure of the capability of a polymer in solution to enhance the viscosity of the solution. Intrinsic viscosity increases with increasing polymer molecular weight, is a function of polymerization conditions, and can be varied independently of the L/G ratio of the polymer.

### Chronic Pain and Fibromyalgia

Pain is a sensation and a perception that is comprised of a complex series of mechanisms. Pain can be experienced both acutely and chronically. Acute pain is the instantaneous onset of a painful sensation as a self-warning to living organisms and chronically developing pains as an illness. Unlike acute pain (e.g., the transient protective physiology pain), chronic pain usually has a delayed onset but can last for hours to days, or even months or years. Chronic pain can involve an amalgamation of physical, social, and psychologic factors. A non-negligible number of patients suffer intractable diseases for which no therapies have been established because the causal mechanism of onset remains unclear. Among them is generalized pain syndrome. Generalized pain syndrome is a disease of unknown cause that produces intense pain over a wide area of the body, and is difficult to detect ecologically abnormal findings by examinations. Because generalized pain syndrome tends to often become chronic, it is positioned as an intractable chronic pain. It is known that in addition to pain, generalized pain syndrome is often complicated by a sensation of fatigue, depression, anxiety and the like. Fibromyalgia is a form of generalized pain syndrome, which occurs prevalently.

Chronic pain syndromes can be differentiated into 4 main groups: nociceptive pain, which occurs normally in response to noxious stimuli and continues only as long as the stimuli are maintained; inflammatory pain, which results from tissue injury and the subsequent inflammatory response and disappears after resolution of the injury; dysfunctional pain, which is maladaptive, providing neither protection from injury nor support for the healing and repair processes; and neuropathic pain, which can follow damage either to peripheral neurons (e.g., from mechanical trauma, metabolic disease, neurotoxic chemicals, herpes zoster and other infections, or tumor invasion) or those in the CNS (from cord injury, stroke, or multiple sclerosis). Thus, different types of pains have different symptoms and causes. For example, fibromyalgia and complex regional pain syndrome (CRPS) appear to share a common pathophysiologic mechanism-supersensitivity of brain mechanisms that receive and integrate nociceptive signals-and associated with the activities of the N-methyl-D-aspartate (NMDA) receptors for brain glutamate. However, fibromyalgia is usually included among the dysfunctional pain group and reflects malfunctioning sensory processing within the CNS, in absent of prior tissue damage; complex regional pain syndrome (CRPS) often occurs at sites of a prior injury or surgical procedure.

The chronic pain can be neurological pain, neuropathies, polyneuropathies, diabetes-related polyneuropathies, idiopathic chronic pain (e.g., juvenile fibromyalgia syndrome (JFMS)), trauma, migraine, tension headache, cluster headache, Horton's disease, varicose ulcers, neuralgias, musculo-skeletal pain, complex regional pain syndrome (CRPS), osteo-traumatic pain, fractures, algodystrophy, spondyloarthritis, fibromyalgia, phantom limb pain, back pain, vertebral pain, post-surgery pain, herniated intervertebral disc-induced sciatica, cancer-related pain, vascular pain, visceral pain, or pain from childbirth.

### Fibromyalgia

Fibromyalgia is a chronic disease involving widespread pain, stiffness and tenderness in musculoskeletal-related tissues including muscles, tendons and ligaments. Patients with fibromyalgia show sleep disturbances, fatigue, anxiety and/or fibrofog. Fibrofog encompasses the inability to concentrate memory loss and depression. Fibromyalgia occurs in approximately 2% of the United States general population with a higher incidence in women (3.4%) compared to men (0.5%).

The most widely used criteria for diagnosing fibromyalgia—those proposed by the American College of Rheumatology—require only that patients complain of widespread musculoskeletal pain and that they exhibit excessive tenderness when mild pressure (sufficient to cause blanching beneath the physician's fingernail) is applied at 11 or more among 18 predetermined anatomical sites. The core symptom of fibromyalgia is the widespread pain described as arising from muscles and joints. The loci at which fibromyalgia pain is felt are not usually those of prior tissue damage. Many patients with fibromyalgia also have tender skin. The pain typically increases and decreases in intensity with flares accompanying unusual exertion, prolonged inactivity, soft tissue injuries, surgery, poor sleep, cold exposure, long car trips and stress. Pain is predominately axial in location but can also occur in hands and feet.

Fibromyalgia patients exhibit neither physical signs nor laboratory evidence of any distinct pathologic process, which may be because its pathogenesis as reflecting disordered central pain processing. Fibromyalgia has been shown to be comorbid with bipolar disorder, major depressive disorder, any anxiety disorder, any eating disorder and any substance abuse disorder. These diseases may share underlying pathophysiological links.

Juvenile fibromyalgia syndrome (JFMS) is an idiopathic chronic pain syndrome that occurs in children and adolescents, particularly teenage girls. JFMS is associated with extra-articular musculoskeletal system pain and specific tender points (TPs). Additional common symptoms include sleeping difficulties, fatigue, irritable bowel syndrome, mood swings, subjective soft tissue swelling and headaches. JFMS begins in youngsters and is increasingly diagnosed in adolescents. At present, 2-6.1% of school-age children are thought to have JFMS.

LYRICA (pregabalin) Capsules, CV is approved for maintenance treatment of fibromyalgia. Antidepressants duloxetine (Cymbalta) and milnacipran (Savella) are also approved for treatment of fibromyalgia. Cymbalta is approved for the treatment of fibromyalgia in adult and in pediatric patients aged 13-17 years old. However, published animal studies demonstrate that the administration of anesthetic and sedation drugs that block NMDA receptors and/or potentiate GABA activity increases neuronal apoptosis in the developing brain and results in long-term cognitive deficits, when used for longer than 3 hours. The clinical significance of these findings is not clear. However, based on the available data, the window of vulnerability to these changes is believed to correlate with exposures in the third trimester of gestation through the first several months of life, but may extend out to approximately three years of age in humans. Some published studies in children suggest that similar deficits may occur after repeated or prolonged exposures to anesthetic agents early in life and may result in adverse cognitive or behavioral effects. Savella is not approved for use in pediatrics.

### Complex Regional Pain Syndrome (CRPS)

CRPS is another type of chronic pain, which usually (but not always) occurs at sites of a prior injury or surgical procedure. CRPS is more common in women (80%) and first appears at any age, which is characterized by severe neuropathic pain usually preceded by an injury (77% of cases) or surgical procedure (11%), but sometimes occurring without antecedent tissue damage. The pain is usually described as burning and aching, and exhausting. Among patients in whom the pain does not remit spontaneously, its intensity tends not to decrease with time and may worsen. Most patients (84%-90%) exhibit touch allodynia (e.g., enhanced pain when touched or lightly brushed) and static mechanoallodynia (91%-96%) (pain when light pressure is applied); half or more also describe visceral pain.

### Other Types of Chronic Pain

The pain can be a symptom associated with a disorder suffered by the subject. Chronic pain can be associated with conditions such as inflammation, nerve injury, cancer, surgery, bone fracture, tumor metastasis, osteoarthritis, arthritis (e.g., psoriatic arthritis, rheumatoid arthritis), multiple sclerosis, stomach ulcers, fungal infections, bacterial infections, viral infections (e.g., AIDS, hepatitis), gallbladder disease, diabetes interstitial cystitis, and chronic pancreatitis. Chronic pain occurs in a variety of forms including, but not limited to spontaneous pain (painful sensation without an external stimulus), allodynia (painful sensation in response to a normally innocuous stimulus) and hyperalgesia (strong painful sensation to a mildly painful stimulus). Persistent pain can be caused by many different factors. For example, persistent pain can be caused by conditions that accompany the aging process (for example conditions that may affect bones and joints in ways that cause persistent pain). As another example, persistent pain may also be caused by conditions, such as rheumatoid arthritis, osteoarthritis, cancer, multiple sclerosis, stomach ulcers, fungal infections, bacterial infections, viral infections (such as AIDS, hepatitis), and gallbladder disease. In some embodiments, persistent pain can be caused by inflammation or nerve injury (for example, damage to or malfunction of the nervous system). In some embodiments, persistent pain can be inflammatory pain or neuropathic pain (for example, peripheral neuropathic pain and central neuropathic pain). In some embodiments, persistent pain is mediated by hyper-excitable pain-processing neurons in peripheral and central nervous system (e.g., peripheral sensitization, central sensitization).

### N-methyl-D-aspartate (NMDA) Receptor and Ketamine

### NMDA Receptor

An NMDA receptor is a postsynaptic, ionotropic receptor that is responsive to, inter alia, the excitatory amino acids glutamate and glycine and the synthetic compound NMDA. The NMDA receptor controls the flow of both divalent and monovalent ions into the postsynaptic neural cell through a receptor associated channel. Activation of the NMDA receptor has been shown to be the central event which leads to excitotoxicity and neuronal death in many disease states, as well as a result of hypoxia and ischemia following head trauma, stroke and following cardiac arrest. The NMDA receptor has been implicated during development in specifying neuronal architecture and synaptic connectivity, and may be involved in experience-dependent synaptic modifications. In addition, NMDA receptors are also thought to be involved in long term potentiation and central nervous system disorders.

NMDA receptor antagonists are therapeutically valuable for a number of reasons. In addition to anesthesia, certain NMDA receptor antagonists confer profound analgesia, a highly desirable component of general anesthesia and sedation. Moreover, NMDA receptor antagonists are neuroprotective under many clinically relevant circumstances (including neuropathic pain states, ischemia, brain trauma, and certain types of convulsions).

Commercially available NMDA antagonists have a wide variety of uses. For example, ketamine has been used as an anesthetic agent. There are numerous applications for NMDA antagonist formulations without neurotoxicity in supervised medical practice. Ketamine, for example, can be used as an analgesic for breakthrough pain, anesthesia and sedation.

### Ketamine and Drug Product

The chemical structure ketamine (chemical formula C13H16CINO, IUPAC name 2-(2-chlorophenyl)-2-(methylamino) cyclohexan-1-one) is shown below.

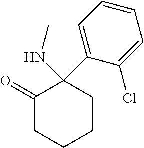 [SMILES: CNC1(c2ccccc2Cl)CCCCC1=O]

The chemical structure ketamine hydrochloride (chemical formula C₁₃H₁₇C₁₂NO, IUPAC name 2-(2-chlorophenyl)-2-(methylamino) cyclohexan-1-one hydrochloride) is shown below.

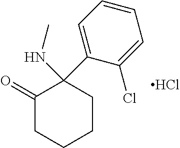 [SMILES: CNC1(c2ccccc2Cl)CCCCC1=O ; Cl]

Ketamine hydrochloride has a molecular weight of 74.18 and solubility in water of 200 mg/mL. It is freely soluble in methanol and soluble in ethanol (˜750 mg/mL), but practically insoluble in ether. Its Log P is 2.2 and pKa is 7.5.

Ketamine hydrochloride is approved for parenteral administration (intravenous or intramuscular) for the induction of anesthesia (e.g., Ketalar (ketamine hydrochloride) injection, NDA 016812). Ketamine is a racemic mixture of two enantiomers; the S-(+)-ketamine is considered the more potent form (esketamine hydrochloride) and is also approved as a nasal spray product for the treatment of treatment-resistant depression (TRD) in adults (e.g., Spravato (esketamine hydrochloride) spray, NDA 211243). The composition disclosed herein can comprise enantiomerically pure S-(+)-ketamine (also known as esketamine), enantiomerically pure R-(−)-ketamine (also known as arketamine), or a racemic mixture of S-(+)-ketamine and R-(−)-ketamine. The composition can comprise ketamine hydrochloride.

KETALAR (ketamine hydrochloride), which is approved for intravenous or intramuscular injection, is a general anesthetic indicated as the sole anesthetic agent for diagnostic and surgical procedures that do not require skeletal muscle relaxation and for the induction of anesthesia prior to the administration of other general anesthetic agents as a supplement to other anesthetic agents. Induction of anesthesia can be achieved through intravenous route (Initially, 1 to 4.5 mg/kg administered slowly over a period of 60 seconds. Alternatively, administer a dose of 1 to 2 mg/kg at a rate of 0.5 mg/kg/min) or intramuscular route (Initially, 6.5 to 13 mg/kg). Maintenance of anesthesia may be achieved through increments of one-half to the full induction dose, repeated as needed. The dose may be adjusted according to the patient's anesthetic needs and whether an additional anesthetic agent is employed. The regimen of a reduced dose of KETALAR supplemented with diazepam can be used to produce balanced anesthesia by combination with other agents.

SPRAVATO™ (esketamine) nasal spray, CIII is a non-competitive NMDA receptor antagonist indicated, in conjunction with an oral antidepressant, for the treatment of treatment-resistant depression (TRD) in adults. The dose of SPRAVATO™ is 28 mg of esketamine per device. Each nasal spray device delivers two sprays containing a total of 28 mg of esketamine.

### Mechanism of Action

A racemic mixture of ketamine, is a non-selective, non-competitive antagonist of the N-methyl-D-aspartate (NMDA) receptor, an ionotropic glutamate receptor. The major circulating metabolite of ketamine (e.g., norketamine) demonstrated activity at the same receptor with less affinity. Norketamine is about ⅓ as active as ketamine in reducing halothane requirements (MAC) of the rat. Ketamine interacts with NMDA receptors, opioid receptors, monoaminergic receptors, muscarinic receptors and voltage sensitive Ca ion channels. Unlike other general anesthetic agents, ketamine does not interact with GABA receptors.

### Methods for Treating Fibromyalgia and Ameliorating Pain

Disclosed herein include methods for treating pain (e.g., fibromyalgia) in a subject in need thereof. In some embodiments, the method comprises administering to the subject a composition comprising at least one selected from the group consisting of ketamine, norketamine and a pharmaceutically acceptable salt thereof.

The subject can be administrated at a dose of 0.01 mg/kg-10 mg/kg (e.g., from about 0.05 mg/kg to about 1 mg/kg, from about 0.1 mg/kg to about 0.5 mg/kg, from about 0.05 mg/kg to about 0.5 mg/kg, from about 1 mg/kg to about 3 mg/kg, or from about 2 mg/kg to about 5 mg/kg) of the active ingredient, depending upon the route of administration. The subject can be administrated intravenously at the dose of 0.1 mg/kg, 0.3 mg/kg or 0.5 mg/kg.

The subject can be administrated through oral administration, sublingual administration, injection, topical administration, local infiltration, rectal administration or intranasal administration. The injection can be intravenous, intramuscular, subcutaneous, intraperitoneal, intrathecal, or intra-rectally. The local infiltration can be infiltration into the skin, mucosa, epithelia or organs. In some embodiments, the implant is administrated to a subject via a single subcutaneous injection. The injection can be conducted at a healthcare facility (e.g., a hospital) or at home by the patient.

Disclosed herein include methods for treating fibromyalgia in a subject in need thereof. In some embodiments, the method comprises administering to the subject an implant comprising a composition comprising at least one selected from the group consisting of ketamine, norketamine and a pharmaceutically acceptable salt thereof.

In some embodiments, the implant of methods disclosed herein is a sterile implant. In some embodiments, the implant need not to be sterile. The implant can be substantially sterile, have been sterilized, or be sterile, except for minor contamination introduced between removal from the sterile wrapper and implantation.

The implant can further comprise a pharmaceutically acceptable excipient. The excipient can be polymer, microsphere matrix, monosodium glutamate (MSG), magnesium chloride (MgCl₂), sugars, oils, lipids, and carbohydrates. Pharmaceutical compositions according to the invention can be formulated to release the active compound substantially immediately upon administration or at any predetermined time or time period after administration. The latter types of compositions are generally known as controlled release formulations (also known as extended-release formulations), which include (i) formulations that create a substantially constant concentration of the active compound within the body over an extended period of time; (ii) formulations that after a predetermined lag time create a substantially constant concentration of the active compound within the body over an extended period of time; and (iii) formulations that sustain active compound action during a predetermined time period by maintaining a relatively, constant, effective active compound level in the body with concomitant minimization of undesirable side effects associated with fluctuations in the plasma level of the active compound (sawtooth kinetic pattern).

Administration of the active compound in the form of a controlled release formulation is especially preferred in cases in which the active compound, either alone or in combination with a second agent, at therapeutic levels produces unwanted side effects, such as nausea.

As discussed herein, the implant comprising ketamine, norketamine and/or a pharmaceutically acceptable salt thereof can be an injectable formulation comprising a biodegradable polymer (e.g., polyester poly (D,L-lactide-co-glycolide) (PLGA)). The PLGA-containing composition, in some embodiments, is an injectable implant, and/or is capable of extended release of ketamine.

Disclosed herein include methods for treating fibromyalgia in a subject in need thereof. In some embodiments, the method comprises administering to the subject an implant comprising (i) a composition comprising at least one selected from the group consisting of ketamine, norketamine and a pharmaceutically acceptable salt thereof, and (ii) a polymer (e.g., PLGA). The polymer can be a biodegradable polymer such as poly (lactide) (PLA) or PLGA. PLGA is a copolymer of poly lactic acid (PLA) and poly glycolic acid (PGA). Poly lactic acid contains an asymmetric a-carbon which is typically described as the D or L form in classical stereochemical terms and sometimes as R and S form, respectively. As used herein, the term “poly (lactide) polymer” does not encompass a poly (lactic-co-glycolic acid) polymer. In some embodiments, suitable biodegradable polymers for the implant disclosed herein comprise PLGA copolymers, polyesteramides, polyanhydrides, polyacetals, polycaprolactones, polycarbonates, or any combination thereof. In some embodiments, the biodegradable polymer comprises a PLGA copolymer.

The characteristics of PLGA are controlled by the stereochemistry of lactic acid (D, L, or DL), degree of crystallinity, lactic acid/glycolic acid ratio, and molecular weight. For example, high inherent viscosity compromises the diffusion of encapsulated drugs, therefore favors the encapsulation efficiency. Lactide-rich PLGA is more hydrophobic than counterparts with a high glycolic ratio, showing much slower degradation due to abundant methyl side groups. Exceptionally, the copolymer of 50:50 ratio exhibits the fastest degradation, which is probably ascribed to its lowest crystallinity as well as excellent hydrophilicity. In some embodiments, the biodegradable polymer has an average molecular weight from about 30 kDa to about 300 kDa.

PLGA polymers are commercially available with different terminal groups, namely, free carboxylic acid (COOH) end groups (uncapped) or esterified terminal groups (capped). The end groups of PLGA can influence drug encapsulation efficiency, degradation, stability, and conjugation of ligands. For example, COOH terminated PLGA polymers can result in a slightly acidic environment, affecting the solubility of the drugs released from the polymers. In some embodiments, the biodegradable polymers are ester end-capped (ester-terminated). In some embodiments, the biodegradable polymers are uncapped (acid-terminated).

In some embodiment, the implants disclosed herein exhibit the advantage that the internal pH environment drops as the polymer degrades to constituent monomers. The drop in pH upon degradation can improve the time-dependent release of drugs and active agents (e.g., ketamine) that are insoluble or less soluble at neutral pH (and thus locked in the implant), but become increasingly soluble as pH drops. In some embodiments, the implants improve release of drugs (e.g., ketamine) with increased solubility at low pH. In some embodiments, the implants improve release of drugs with an acidic pKa. In some embodiments, the increased time-dependent release increases ability of the compound (e.g., ketamine) to be released into the systemic circulation. In some embodiments, the drop in pH upon degradation increases the rate of degradation of the polymer with respect to time. In some embodiments, the drop in pH upon degradation results in auto-catalysis of degradation of the polymer.

In some embodiments, the PLGA copolymer has a co-monomer ratio for lactide to glycolide (L/G) content of about 50:50 to 100:0 or 50:50 to about 85:15. In some embodiments, the PLGA copolymer has a co-monomer ratio for lactide to glycolide (L/G) content of 50:50 or 75:25. The co-monomer ratio for L/G content can be molar ratio, volume ratio or weight ratio.

In some embodiments, the molar ratio for L/G content of the polymer is between 50:50 and 100:0. In some embodiments, the ratio is between 50:50 and 55:45, between 55:15 and 60:40, between 60:40 and 65:35, between 65:35 and 70:30, between 70:30 and 75:25, between 75:25 and 80:20, between 80:20 and 85:15, between 85:15 and 90:10, between 90:10 and 95:5, between 95:5 and 100:0, or a range between any two of the values. In some embodiments, the ratio is 50:50, 52:48, 54:46, 56:44, 58:42, 60:40, 62:38, 64:36, 66:34, 68:32, 70:30, 72:28, 74:26, 76:24, 78:22, 80:20, 82:18, 84:16, 86:14, 88:12, 90:10, 92:8, 94:6, 96:4. In another embodiment, the ratio is 97:3, 98:2, 99:1, 100:0 (e.g. substantially less than 1% glycolide), or a number between any two of the values. In some embodiments, the ratio is 50:50. In some embodiments, the ratio is 85:15.

In some embodiments, the polymer disclosed herein exhibits an inherent viscosity of between about 0.1-2 dl/g in chloroform. In some embodiments, the inherent viscosity is 0.1-0.2 dl/g, 0.15-0.25 dl/g, 0.2-0.3 dl/g, 0.25-0.35 dl/g, 0.3-0.4 dl/g, 0.35-0.45 dl/g, 0.4-0.5 dl/g, 0.45-0.55 dl/g, 0.5-0.6 dl/g, 0.55-0.65 dl/g, 0.6-0.7 dl/g, 0.65-0.75 dl/g, 0.7-0.8 dl/g, 0.75-0.85 dl/g, 0.8-0.9 dl/g, 0.85-0.95 dl/g, 0.9-1.0 dl/g, 0.95-1.05 dl/g, 1.0-1.1 dl/g, 1.05-1.15 dl/g, 1.1-1.2 dl/g, 1.15-1.25 dl/g, 1.2-1.3 dl/g, 1.25-1.35 dl/g, 1.3-1.4 dl/g, 1.35-1.45 dl/g, 1.4-1.5 dl/g, 1.45-1.55 dl/g, 1.5-1.6 dl/g, 1.55-1.65 dl/g, 1.6-1.7 dl/g, 1.65-1.75 dl/g, 1.7-1.8 dl/g, 1.75-1.85 dl/g, 1.8-1.9 dl/g, 1.85-1.95 dl/g, or 1.9-2.0 dl/g, or a range between any two of the values. In some embodiments, the inherent viscosity is 0.4-0.6 dl/g. In some embodiments, the inherent viscosity is 0.1 dl/g, 0.15 dl/g, 0.2 dl/g, 0.25 dl/g, 0.3 dl/g, 0.35 dl/g, 0.4 dl/g, 0.45 dl/g, 0.5 dl/g, 0.55 dl/g, 0.6 dl/g, 0.65 dl/g, 0.7 dl/g, 0.75 dl/g, 0.8 dl/g, 0.85 dl/g, 0.9 dl/g, 0.95 dl/g, 1.0 dl/g, 1.05 dl/g, 1.1 dl/g, 1.15 dl/g, 1.2 dl/g, 1.25 dl/g, 1.3 dl/g, 1.35 dl/g, 1.4 dl/g, 1.45 dl/g, 1.5 dl/g, 1.55 dl/g, 1.6 dl/g, 1.65 dl/g, 1.7 dl/g, 1.75 dl/g, 1.8 dl/g, 1.85 dl/g, 1.9 dl/g, 1.95 dl/g, 2.0 dl/g, or a number between any two of the values.

In some embodiments, the implant disclosed herein has a surface area to volume (SA:V) ratio between about 0.5 and 3 (millimeters [mm]) 2/mm³.In some embodiments, the ratio is 0.5-1 mm²/mm³, 0.7-1.2 mm²/mm³, 0.9-1.4 mm²/mm³, 1.1-1.6 mm²/mm³, 1.3-1.8 mm²/mm³, 1.5-2 mm²/mm³, 2-2.5 mm²/mm³, 2.5-3 mm²/mm³, 3-3.5 mm²/mm³, 3.5-4 mm²/mm³, 4-4.5 mm²/mm³, 4.5-5 mm²/mm³, 5-5.5 mm²/mm³, 5.5-6 mm²/mm³, 0.5-1.5 mm²/mm³, 1-2 mm²/mm³, 1.5-2.5 mm²/mm³, 2-3 mm²/mm³, 2.5-3.5 mm²/mm³, 3-4 mm²/mm³, 3.5-4.5 mm²/mm³, 4-5 mm²/mm³, 4.5-5.5 mm²/mm³, 5-6 mm²/mm³, 5.5-6.5 mm²/mm³, 6-7 mm²/mm³, 6.5-7.5 mm²/mm³, 7-8 mm²/mm³, 0.5-2 mm²/mm³, 1-2.5 mm²/mm³, 1.5-3 mm²/mm³, 2-3.5 mm²/mm³, 2.5-4 mm²/mm³, 3-4.5 mm²/mm³, 3.5-5 mm²/mm³, 4-5.5 mm²/mm³, 4.5-6 mm²/mm³, 5-6.5 mm²/mm³, 5.5-7 mm²/mm³, 6-7.5 mm²/mm³, or 6.5-8 mm²/mm³, or a range between any two of the values. In some embodiments, the ratio is 0.5 mm²/mm³, 0.6 mm²/mm³, 0.7 mm²/mm³, 0.8 mm²/mm³, 1.0 mm²/mm³, 1.5 mm²/mm³, 2 mm²/mm³, 2.5 mm²/mm³, 3 mm²/mm³, 3.5 mm²/mm³, 4 mm²/mm³, 4.5 mm²/mm³, 5 mm²/mm³, 5.5 mm²/mm³, 6 mm²/mm³, 6.5 mm²/mm³, 7 mm²/mm³, or a number between any two of the values.

In some embodiments, the drug (e.g., ketamine) load of an implant disclosed herein is between 1%-99%. In some embodiments, the drug load is between about 1-5%, 2-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-99%, or a range between any two of the values. In some embodiments, the drug load is 2%, 3%, 5%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 65%, 70%, or a number between any two of the values. In some embodiments, ketamine, norketamine and a pharmaceutically acceptable salt thereof is present in an amount of 10%-50% of the mass of the implant. In some embodiments, ketamine, norketamine and a pharmaceutically acceptable salt thereof is present in an amount of 20% or 40% of the mass of the implant.

In some embodiments, the polymer (e.g., PLGA) is present in an amount of between 1%-99% of the mass of the implant. In some embodiments, the polymer (e.g., PLGA) is present in an amount of between about 1-5%, 2-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-99%, or a range between any two of the values of the mass of the implant. In some embodiments, the polymer (e.g., PLGA) is present in an amount of 2%, 3%, 5%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 65%, 70%, or a number between any two of the values of the mass of the implant. In some embodiments, PLGA is present in an amount of 60% of the mass of the implant. In some embodiments, PLGA is present in an amount of 80% of the mass of the implant.

In some embodiments, the implant of methods disclosed herein is rod shaped. In some embodiments, the implant is disc shaped, cylindrical, sheet-shaped or any shape suitable for retention in a body tissue (e.g. subcutaneous tissue). Rod-shaped, disk-shaped, cylindrical, sheet shaped implants can be used to provide extended delivery of ketamine, norketamine and a pharmaceutically acceptable salt thereof.

In some embodiments, the shape of cross-section of the rod-shaped implant is substantially round. In some embodiments, the longest diameter of the substantially round cross-section is less than 150%, less than 145%, less than 140%, less than 135%, less than 130%, less than 125%, less than 120%, less than 115%, less than 110%, less than 105%, or a number or a range between any two of the values of its shortest diameter. The ratio of the longest to the shortest diameter can be any other ratio consistence with a substantially round shape. The cross-sectional shape can also be any other cross-sectional shape (e.g., rectangular or square cross-sectional shape). In some embodiments, the cross-section of the rod-shaped implant is round (e.g., the longest diameter is 100% of the values of its shortest diameter). In some embodiments, the diameter is 0.1-0.5 mm, 0.5-1 mm, 1-1.5 mm, 1.5-2 mm, 2-2.5 mm, 2.5-3 mm, 3-3.5 mm, 3.5-4 mm, 4-4.5 mm, 4.5-5 mm, or a range between any two of the values. In some embodiments, the diameter is 0.1 mm, 0.2 mm, 0.4 mm, 0.6 mm, 0.8 mm, 1 mm, 1.2mm, 1.4 mm, 1.6 mm, 1.8 mm, 2 mm, 2.2 mm, 2.4 mm, 2.6 mm, 2.8 mm, 3 mm, 3.2 mm, 3.4mm, 3.6 mm, 3.8 mm, 4 mm, 4.2 mm, 4.4 mm, 4.6 mm, 4.8 mm, 5 mm, or a number between any two of the values. In some embodiments, the implant has a diameter of about 1.2 mm.

In some embodiments, the cross-sectional area is substantially constant over the length of the rods and cylinders, and thickness of disks of the present invention. In some embodiments, the cross-sectional area is not constant. In some embodiments, the cross-sectional dimensions are substantially constant over the length of the rods, disks, and cylinders of the present invention. In some embodiments, the cross-sectional dimensions are not constant.

In some embodiments, the length of the rod-shaped implant is at least twice the diameter of the cross-section. In some embodiments, the length is at least as great as the diameter of the cross-section. In some embodiments, the length is at least 1.1 times, at least 1.2 times, at least 1.3 times, at least 1.4 times, at least 1.5 times, at least 1.6 times, at least 1.7 times, at least 1.8 times, at least 1.9 times, at least 2.2 times, at least 2.5 times, at least 3 times, at least 4 times or a range between any two of the values the diameter or a number. In some embodiments, the implant has a length between about 1-50 mm. In some embodiments, the length of the implant is 1-1.5 mm, 1.5-2 mm, 2-2.5 mm, 2.5-3 mm, 3-3.5 mm, 3.5-4 mm, 4-4.5 mm, 4.5-5 mm, 5-5.5 mm, 5.5-6 mm, 6-6.5 mm, 6.5-7 mm, 7-7.5 mm, 7.5-8 mm, 8-8.5 mm, 8.5-9 mm, 9-9.5 mm, 9.5-10 mm, 10-10.5 mm, 10.5-11 mm, 11-11.5 mm, 11.5-12 mm, 12-12.5 mm, 12.5-13 mm, 13-13.5 mm, 13.5-14 mm, 14-14.5 mm, 14.5-15 mm, 15-15.5 mm, 15.5-16 mm, 16-16.5 mm, 16.5-17 mm, 17-17.5 mm, 17.5-18 mm, 18-18.5 mm, 18.5-19 mm, 19-19.5 mm, 19.5-20 mm, 20-20.5 mm, 20.5-21 mm, 21-21.5 mm, 21.5-22 mm, 22-22.5 mm, 22.5-23 mm, 23-23.5 mm, 23.5-24 mm, 24-24.5 mm, 24.5-25 mm, 25-25.5 mm, 25.5-26 mm, 26-26.5 mm, 26.5-27 mm, 27-27.5 mm, 27.5-28 mm, 28-28.5 mm, 28.5-29 mm, 29-29.5 mm, 29.5-30 mm, 30-30.5 mm, 30.5-31 mm, 31-31.5 mm, 31.5-32 mm, 32-32.5 mm, 32.5-33 mm, 33-33.5 mm, 33.5-34 mm, 34-34.5 mm, 34.5-35 mm, 35-35.5 mm, 35.5-36 mm, 36-36.5 mm, 36.5-37 mm, 37-37.5 mm, 37.5-38 mm, 38-38.5 mm, 38.5-39 mm, 39-39.5 mm, 39.5-40 mm, 40-40.5 mm, 40.5-41 mm, 41-41.5 mm, 41.5-42 mm, 42-42.5 mm, 42.5-43 mm, 43-43.5 mm, 43.5-44 mm, 44-44.5 mm, 44.5-45 mm, 45-45.5 mm, 45.5-46 mm, 46-46.5 mm, 46.5-47 mm, 47-47.5 mm, 47.5-48 mm, 48-48.5 mm, 48.5-49 mm, 49-49.5 mm, or 49.5-50 mm, or a range between any two of the values. In some embodiments, the length is 1.0 mm, 1.2 mm, 1.4 mm, 1.6 mm, 1.8 mm, 2.0 mm, 2.2 mm, 2.4 mm, 2.6 mm, 2.8 mm, 3.0 mm, 3.5 mm, 4 mm, 4.5 mm, 5 mm, 5.5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, or a number between any two of the values. In some embodiments, the length is about 20 mm.

In some embodiments, the disk-shaped implant has a substantially round, flat shape. In some embodiments, the shape is oval, square, rectangular, etc. The thickness can be less than the diameter of the circle, oval, etc. In some embodiments, the thickness is less than 0.9 times, less than 0.8 times, less than 0.7 times, less than 0.6 times, less than 0.5 times, less than 0.4 times, less than 0.3 times, less than 0.2 times, or less than 0.1 times the diameter.

In some embodiments, the implant has a mass of 0.1-3 g. In some embodiments, the implant has a mass of 0.1-0.5 g, 0.5-1 g, 1-1.5 g, 1.5-2 g, 2-2.5 g, 2.5-3 g, or a number between any two of the values. In some embodiments, the mass is 0.1 g, 0.2 g, 0.3 g, 0.4 g, 0.5 g, 0.6 g, 0.7 g, 0.8 g, 0.9 g, 1 g, 1.1 g, 1.2 g, 1.3 g, 1.4 g, 1.5 g, 1.6 g, 1.7 g, 1.8 g, 1.9g, 2 g, 2.1 g, 2.2 g, 2.3 g, 2.4 g, 2.5 g, 2.6 g, 2.7 g, 2.8 g, 2.9 g, or 3 g.

In some embodiments, the implant is manufactured by a process comprising solvent casting, compression molding, melt-mixing, a melt mix extrusion method that does not require use of either a surfactant or an emulsion or both, or extrusion molding. In some embodiments, the extrusion molding is high-pressure extrusion molding. In some embodiments, implants manufactured by compression molding exhibit increased density. In some embodiments, implants manufactured by compression molding exhibit improved uniformity. In some embodiments, a greater variety of shapes of implants can be manufactured by compression molding. In some embodiments, less material is lost during fabrication in the case of implants manufactured by extruding.

In some embodiments, the implants are stable at body temperature for the delivery period. In some embodiments, the implants are completely eroded or degraded, thus exhibiting a lack of necessity of removing residual material. In some embodiment, the erosion is primarily surface erosion, bulk erosion or a combination of both.

In some embodiments, the implant is biodegradable. In one embodiment, the half-life of the implant in a living organism is 1 month or less, 2 months or less, 3 months or less, 4 months or less, 5 months or less, 6 months or less, 7 months or less, 8 months or less, 9 months or less, 10 months or less, one year or less, 1.5 years or less, 2 years or less, 3 years or less, 4 years or less, 5 years or less, 7 years or less or 10 years or less.

In some embodiments, the composition is released from the polymer over an extended period of time. In some embodiments, the extended period of time is at least 14 days. In some embodiments, the extended period of time is at least 30 days. The time period over which the therapeutic level of a drug is maintained by methods disclosed herein can be at least 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, or 18 months.

Methods of insertion of implants are well known in the art. In some embodiments, implants are inserted through a minimally invasive approach, using a surgical instrument. The rod-shaped implants can provide an advantage due to their ease of implantation and lack of subsequent discomfort. An advantage of rod-shaped implants can be the small incisions (e.g., about 2 mm, 3 mm, 4 mm, 5 mm, 6 mm, or 7 mm) required for their insertion. Another advantage can be due to their ability to be implanted on an outpatient basis. The incision site can be closed with either a single stitch or steristrips. The implant can also be inserted by any other surgical method known in the art.

In some embodiments, the implants disclosed herein provide an advantage due to their lack of necessity of the subject receiving administration every few days or weeks. In some embodiments, an implant is administered again after a period of time. In some embodiments, the implant is administered again after about 1 month, about 2 months, about 3 months, about 4 months, about 5 months, about 6 months, about 7 months, about 8 months, about 9 months, about 10 months, about 11 months, about 12 months, about 14 months, about 16 months, about 18 months, about 20 months, about 22 months, about 24 months, about 30 months, or about 36 months.

In some embodiments, the method disclosed herein provides a method for releasing a therapeutic drug (e.g., ketamine) at a substantially linear rate for an extended period of time (e.g., several months) into a body tissue of a subject. In some embodiments, the substantially linear rate is 0.1 mg/day, 0.2 mg/day, 0.3 mg/day, 0.4 mg/day, 0.5 mg/day, 0.6 mg/day, 0.8 mg/day, 1 mg/day, 1.2 mg/day, 1.5 mg/day, 1.8 mg/day, 2.0 mg/day, 2.5 mg/day, 3 mg/day, 3.5 mg/day, 4 mg/day, 5 mg/day, 6 mg/day, 7 mg/day, 8 mg/day, or 10 mg/day. In some embodiments, the rate is 0.1-0.3 mg/day, 0.2-0.4 mg/day, 0.3-0.5 mg/day, 0.4-0.6 mg/day, 0.5-0.7 mg/day, 0.6-0.8 mg/day, 0.7-0.9 mg/day, 0.8-1.0 mg/day, 1.0-1.2 mg/day, 1.2-1.4 mg/day, 1.4-1.6 mg/day, 1.6-1.8 mg/day, 1.8-2 mg/day, 2-2.5 mg/day, 2.5-3 mg/day, 3-3.5 mg/day, 3.5-4 mg/day, 4-4.5 mg/day, 4.5-5 mg/day, 5-5.5 mg/day, or 5.5-6 mg/day.

In some embodiments, the implant is removable, by surgical or other means known in the art. The implant can be removed due to an adverse reaction to the medication therein, a decision by the physician, a decision by the patient, an overdose of medication or any other reason for which the course of treatment is desired to be halted. In some embodiments, the implant is removable throughout the period of drug delivery or the period of detectable drug delivery. In some embodiments, the implant is tethered to assist in locating it and, if necessary, removing it. In some embodiments, following palpation of the implant, a small incision is made and residual material from the implant is retrieved. In another embodiment, the implant is cohesive throughout the period of drug delivery or the period of detectable drug delivery.

The methods disclosed herein can comprise evaluating the symptoms before and/or after the treatment using the methods. The evaluation of the symptoms can comprise evaluation of the pain characteristics, sleep patterns, fatigue severity, psychiatric symptoms, and cognitive dysfunction. Pain characteristics can be evaluated using various methods, tools, scales or indexes, such as 100-mm numerical rating scales, visual analog scales, pain thresholds measured with dolorimetry, pain intensity score, and manual tender point assessment. Symptoms of fibromyalgia can also be evaluated using a Fibromyalgia Impact Questionnaire (FIQ), which measures physical functioning, work status, depression, anxiety, morning tiredness, pain, stiffness, fatigue, and well-being during a particular period of time. The evaluation can be conducted daily, weekly, bi-weekly, monthly, every two months, every three months, every four months, every five months, every six months, or yearly.

The subject can have a reduced pain intensity score, decreased tenderness, increased muscle endurance, decreased pain threshold and/or decreased pain tolerance. The pain intensity score can be reduced by at least 20% (e.g., at least 30%, at least 40%, at least 50%, at least 60%, at least 70%, at least 80%, at least 90%, or at least 99%). The pain intensity score is measured by visual analog scale (VAS). The tenderness, pain threshold and/or pain tolerance can be decreased by at least 20% (e.g., at least 30%, at least 40%, at least 50%, at least 60%, at least 70%, at least 80%, at least 90%, or at least 99%). The muscle endurance can be increased by at least 20% (e.g., at least 30%, at least 40%, at least 50%, at least 60%, at least 70%, at least 80%, at least 90%, or at least 99%).

The method can comprise concurrently administering to the subject a muscle relaxant. The muscle relaxant can be selected from afloqualone, baclofen, carisoprodol, chlormezanone, chlorphenesin carbamate, chlorzoxasozone, cyclobenzaprine, clonazepam, dantrolene, diazepam, eperisone, idrocilamide, inaperisone, mephenesin, mephenoxalone, methocarbamol, metaxalone, mivacurium chloride, orphenadrine, phenprobamate, pridinol mesylate, quinine, tetrazepam, thiocolchicoside, tizanidine, tolperisone, and pharmaceutically acceptable salts thereof. Two or more muscle relaxants can be administered in the same treatment. This combination can be especially useful for situations in which the dominant menstrually related symptoms experienced by the subject include cramping.

The method can further comprise concurrently administering to the subject an anti-inflammatory agent such as naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin. Two or more NSAIDs can be administered in the same treatment. This combination can be especially useful for situations in which the dominant menstrually related symptom experienced by the subject is physical pain.

The method can further comprise concurrently administering to the subject an antidepressant, such as fluoxetine, duloxetine, bupropion, citalopram, escitalopram, paroxetine, lorazepam, fluvoxamine, sertraline, desvenlafaxine, milnacipran, venlafaxine, amitriptyline, nortriptyline, desipramine, alprazolam, agomelatine, etoperidone, or phenelzine. The antidepressant can also be duloxetine or milnacipran. Two or more antidepressants can be administered in the same treatment. This combination can be especially useful for situations in which the dominant menstrually related symptom experienced by the subject is anxiety or dysphoria.

The method can further comprise concurrently administering to the subject an additional therapeutic agent selected from the group consisting of dextromethorphan, and pregabalin.

### Composition and Kits

Compositions for use in treating fibromyalgia are also provided. For example, a composition including ketamine, norketamine and a pharmaceutically acceptable salt thereof for use in treating fibromyalgia in a subject is provided. The administration of ketamine, norketamine and a pharmaceutically acceptable salt thereof results in reduced pain intensity score, decreased tenderness, increased muscle endurance, decreased pain threshold and/or decreased pain tolerance.

The composition can further comprise a pharmaceutically acceptable excipient. The excipient can be polymer, microsphere matrix, monosodium glutamate (MSG), magnesium chloride (MgCl₂), sugars, oils, lipids, and carbohydrates. Pharmaceutical compositions according to the invention can be formulated to release the active compound substantially immediately upon administration or at any predetermined time or time period after administration. The latter types of compositions are generally known as controlled release formulations (also known as extended-release formulations), which include (i) formulations that create a substantially constant concentration of the active compound within the body over an extended period of time; (ii) formulations that after a predetermined lag time create a substantially constant concentration of the active compound within the body over an extended period of time; and (iii) formulations that sustain active compound action during a predetermined time period by maintaining a relatively, constant, effective active compound level in the body with concomitant minimization of undesirable side effects associated with fluctuations in the plasma level of the active compound (sawtooth kinetic pattern).

Administration of the active compound in the form of a controlled release formulation is especially preferred in cases in which the active compound, either alone or in combination with a second agent, at therapeutic levels produces unwanted side effects, such as nausea.

Any of a number of strategies can be pursued in order to obtain controlled release of the active compound in question. In one example, controlled release is obtained by releasing from a polymer over an extended period of time. The polymer can be formulated into an implant. The active ingredient (e.g., ketamine) can be contained in any appropriate amount in any suitable polymer, and is generally present in an amount of 1-95% by weight of the total weight of the composition. The pharmaceutical compositions can be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The extended period of time can be at least 14 days. The extended period of time can be at least 30 days.

Controlled-release compositions for oral use can, e.g., be constructed to release the active compound by controlling the dissolution and/or the diffusion of the active drug substance. Dissolution or diffusion controlled release can be achieved by appropriate coating of a tablet, capsule, pellet, or granulate formulation of compounds, or by incorporating the compound into an appropriate matrix. A controlled-release coating may include one or more of the coating substances, e.g., sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose), shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled-release composition containing one or more of the compounds of the claimed combinations can also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Controlled-release parenteral compositions can be in form of aqueous suspensions, microspheres, microcapsules, magnetic microspheres, oil solutions, oil suspensions, or emulsions. Alternatively, the active drug(s) can be incorporated in biocompatible carriers, liposomes, nanoparticles, implants, or infusion devices. Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)).

The compositions comprising ketamine, norketamine and/or a pharmaceutically acceptable salt thereof as disclosed herein, in some embodiments, can be loaded in a delivery platform comprising a sol-gel/hydrogel based nano-or micro-particles as described in US2020/0030247 (the content of which is hereby expressly incorporated by reference).

As discussed herein, compositions comprising ketamine, norketamine and/or a pharmaceutically acceptable salt thereof can be an injectable formulation comprising a biodegradable polymer (e.g., PLGA). The PLGA-containing composition, in some embodiments, is an injectable implant, and/or is capable of extended release of ketamine. In some embodiments, suitable biodegradable polymers for the compositions disclosed herein comprise PLGA copolymers, polyesteramides, polyanhydrides, polyacetals, polycaprolactones, polycarbonates, or any combination thereof. In some embodiments, the biodegradable polymer comprises a PLGA copolymer having a co-monomer ratio for lactide to glycolide content of about 50:50 to about 85:15. In some embodiments, the biodegradable polymer has an average molecular weight from about 30 kDa to about 300 kDa.

The pharmaceutical composition can be formulated into an implant with the active ingredient (e.g., ketamine) present in an amount of 10%-50% of the mass of the implant and/or the polymer present in an amount of 50%-90% of the mass of the implant. The implant can be rod-shaped or any other suitable shapes with suitable diameters. For example, the implant can be rod-shaped with a length between 5-50 mm (e.g., 20 mm) and a diameter between 0.1-5 mm (e.g., 1.2 mm). The implant can have a mass of about 0.01-1 g (e.g., 0.2-0.8 g). In some embodiments, the biodegradable polymers are ester-terminated or acid-terminated.

In some embodiments, the pharmaceutical composition is stable before transplanted. The term “stable” refers to that the appearance, drug load, and physical and chemical properties of the pharmaceutical composition (e.g., implant) do not change when stored at the temperature and period of time disclosed herein. In some embodiments, the pharmaceutical composition is stable at 0-35° C. (e.g., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., or 35° C.). In some embodiments, the pharmaceutical composition is stable for about or at least about a week, two weeks, one month, two months, three months, four months, six months, one year, two years, three years, or five years. For example, the pharmaceutical composition can be stable at 2,° C., 4° C., 8° C. or 25° C. for a month.

In some embodiments, the implant is stable before transplanted. In some embodiments, the implant is stable at 0-35° C. (e.g., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., or 35° C.). In some embodiments, the implant is stable for about or at least about a week, two weeks, one month, two months, three months, four months, six months, one year, two years, three years, or five years. For example, the implant can be stable at 2, ° C., 4° C., 8° C. or 25° C. for a month.

The controlled release composition (e.g., implant) can have a dissolution profile. The dissolution profile can be evaluated in vitro or in vivo using methods known to one skilled in the art. In some embodiment, the composition has a greater dissolution rate under physiological conditions than under storage conditions. In some embodiments, dissolution profile is the dissolution profile under physiological condition. In some embodiments, no less than 10% of the active ingredient (e.g., ketamine) is released from the composition (e.g., implant) after a day. In some embodiments, no greater than 80% of the active ingredient (e.g., ketamine) is released from the composition (e.g., implant) after a day. In some embodiments, 30%-50% (e.g., 30%, 35%, 40%, 45%, or 50%) of the active ingredient (e.g., ketamine) is released from the composition (e.g., implant) after a day. In some embodiments, no less than 50% of the active ingredient (e.g., ketamine) is released from the composition (e.g., implant) after three day. In some embodiments, no greater than 90% of the active ingredient (e.g., ketamine) is released from the composition (e.g., implant) after three day. In some embodiments, 60%-80% (e.g., 60%, 65%, 70%, 75%, or 80%) of the active ingredient (e.g., ketamine) is released from the composition (e.g., implant) after a day. In some embodiments, no less than 50% (e.g., 50%, 55%, 60%, 65%, 70%, 75%, 80%, 85%, 90%, 95% or 99%) of the active ingredient (e.g., ketamine) is released from the composition (e.g., implant) after a week (e.g., after 7 days, 8 days, 9 days, 10 days, 11 days, 12 days, 13 days, 14 days, 21 days, 1 month, 2 months, 3 months, 4 months, 5 months or 6 months). In some embodiments, the composition (e.g., implant) releases the active ingredient (e.g., ketamine) for at least 1 day (e.g., 1 day, 2 days, 3 days, 4 days, 5 days, 6 days, 7 days, 8 days, 9 days, 10 days, 11 days, 12 days, 13 days, 14 days, 21 days, 1 month, 2 months, 3 months, 4 months, 5 months or 6 months). In some embodiments, the composition (e.g., implant) has a dissolution profile comparable to an implant in the Examples. Dissolution profiles can be compared using a method or model known to one skilled in the art. Methods or models that can compare the similarity between two dissolution profiles can include similarity factor (f₂) and Weibull model. The similarity factor is a logarithmic reciprocal square root transformation of the sum of squared error and is a measurement of the similarity in the percent (%) of dissolution between the two curves/profiles.

Two dissolution profiles are considered similar when the f₂ value is equal to or greater than 50.

Medical kits are also disclosed. The medical kits can include, for example, a dosage supply of ketamine. The active agents can be supplied alone (e.g., lyophilized), or in a pharmaceutical composition. The active agents can be in unit dosage, or in a stock that should be diluted prior to administration. In some embodiments, the kit includes a supply of pharmaceutically acceptable carrier. The kit can also include devices for the administration of the active agents or compositions, for example, pre-filled syringes. The kits can include printed instructions for administering the composition in a use as described above. For instance, the kit comprises ketamine; and a manual providing instructions for administering ketamine to the subject.

### EXAMPLES

Some aspects of the embodiments discussed above are disclosed in further detail in the following examples, which are not in any way intended to limit the scope of the present disclosure.

### Example 1

### Pre-Clinical Extrusion of PLGA/Ketamine Implants

This Example describes the manufacturing of six different preclinical formulations of implants comprising ketamine hydrochloride for the treatment of fibromyalgia. The ketamine hydrochloride loaded implants had drug load at either 20% or 40% drug load with three different bioresorbable polymers (e.g., VitaelR DLG 5005 A, 7505 A, or 7505 E).

Small-batch extrusion trials were conducted to determine drug release and stability of each formulation. The batches were DR-0636, DR-0637, DR-0638, and DR-0639. DR-0636 batch tested drying of the polymers, namely Vitael® DLG 5005 A, 7505 A and 7505 E. DR-0637 batch tested extrusion of DLG 5005 A with 20% and 40% ketamine hydrochloride. DR-0638 batch tested extrusion of DLG 7505 A with 20% and 40% ketamine hydrochloride. DR-0639 batch tested extrusion of DLG 7505 E with 20% and 40% ketamine hydrochloride. The analytical test samples were collected during various stages of the manufacturing process, including the final product. These tests included identifying raw material, inherent viscosity (IV) testing of the conditioned polymers, blend uniformity (BU), IV and assay and related substances (Assay and RS) at the beginning, middle and end of processing and dissolution. The identification of raw material was only performed on the active pharmaceutical ingredient. Various polymers and drug-loading levels of ketamine hydrochloride were evaluated to determine the optimal formulation to proceed forth into pre-clinical trials.

### DR-0636 Drying

To condition the polymers, all three bioresorbable polymers were dried under vacuum. The purpose of conditioning the polymers prior to processing was to avoid any instabilities and physical/mechanical properties problems. Due to their structure and being both polar and hygroscopic, PLGA polymers are subject to absorbing moisture depending on atmospheric conditions. If these polymers were to be processed without being conditioned, the existing moisture would get trapped inside the resin, leading to surface defects and in some cases hydrolysis during subsequent processing. By allowing the polymers to sit under vacuum, with no heat applied, a dry atmosphere was created where moisture dissipated.

Vitael® DLG 5005 A, Vitael DLG 7505 A, and Vitael DLG 7505 E were successfully conditioned from Dec. 19, 2023 to Jan. 29, 2024. The minimum time required to obtain a moisture of ˜0.1% was 48 hours, as a rule of thumb. Conditioning for over 48 hours has not demonstrated any negative impacts on processability nor mechanical/physical properties. After conditioning, 0.5 g of each of the conditioned polymers was sent for IV testing.

### Twin Extrusion

The goal of the twin extrusion trials was to compound and shape ketamine hydrochloride loaded implants to a diameter of 1.19 mm. Prior to extrusion, the conditioned polymers were mixed with the ketamine hydrochloride by using the DAC 330-100 SE speed mixer. This mixer is a dual-asymmetric-centrifuge mixer that is suitable for mixing powders to create homogeneous blends without generating any air pockets. Mixing was conducted at the speed of 1000 rpm for 60 seconds. Approximately 0.5 g of each blend were sent for BU testing. The goal of speed mixing all formulations was to obtain even polymer to ketamine hydrochloride distributions which would be further improved via the extrusion process.

The HAAKE MiniCTW Micro-Conical Twin Screw Compounder was used in a co-rotating configuration to allow for the dispersive mixing of the materials. Additionally, the mini extruder was set up with a 1.0 mm die to allow for the shaping of the final product. Each blend was manually fed in the feed throat of the extruder and the diameter of the extrudate was controlled by controlling the speed of the conveyor. The extrudate was separated into beginning (B), middle (M) and end (E) sections, which represented the time at which extrudate was first collectable, the time at which about half of the blend had been fed, and the time at which there was no more blend to be fed. B, M, and E samples were collected for all formulations and were sent to the lab for IV and Assay and RS. 0.2 g of extruded material from beginning, middle and end were sent for IV.

Finally, extrudate from all formulations was manually cut to about 20 mm to produce 7 samples cut to size for dissolution testing and 30 samples cut to size for storage. 3 implants cut to size from beginning, middle and end, were sent for Assay and RS test. Dimensional analysis was also performed on the 30 samples. The remaining extrudate was packaged in heat-sealed foil bags for preservation.

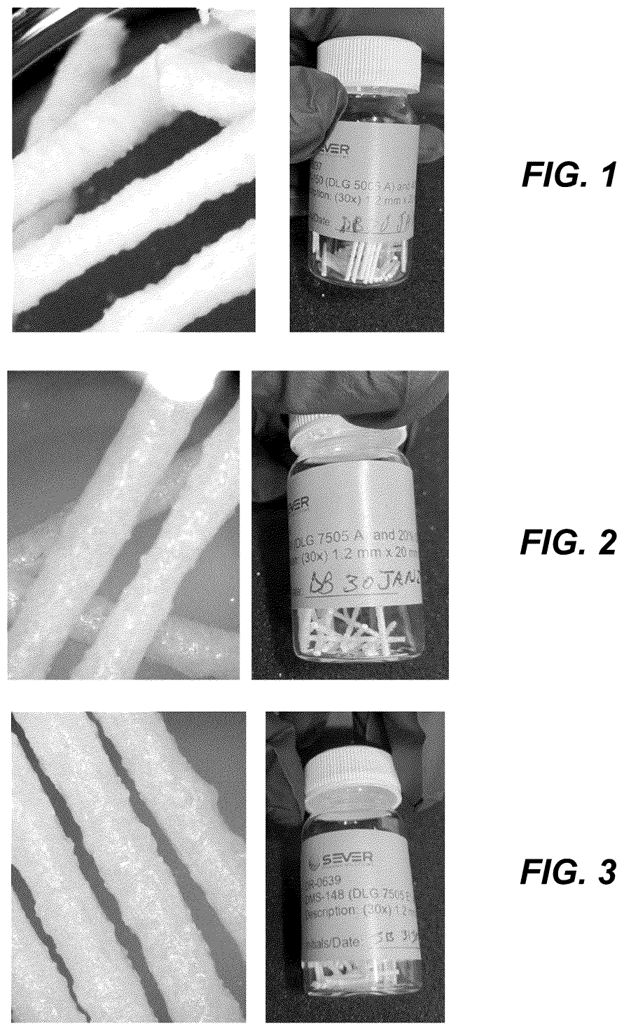

The DR-0637 batch tested extrusion of Vitaelx DLG 5005 A at 20% and 40% drug load. Vitael® DLG 5005 A is an acid terminated biodegradable PLGA copolymer. Its L/G ratio is 50:50, with IV range of 0.4-0.6. The mixing of Vitaelx DLG 5005 A with ketamine hydrochloride at both loadings (e.g., 20% and 40% drug load) went smoothly. The resulting blends visually looked homogeneous, without noticeable clumps. The extrusion parameters were determined based on the melting temperature of VitaelR DLG 5005 A, with temperature set at 130° C. and screw speed set at 25 rpm. The motor load was 0.85 Nm for 20% drug load and 1.08 Nm for 40% drug load. The conveyor belt was set at 1-10 fpm, while the actual values were 8 for both 20% and 40% drug load. The resulting extrudate was visually white and the surface finish looked rough (FIG. 1). From visual observations, it was determined that the shear and temperature of the extruder caused no degradation of the extrudate. The cutting of extrudate went as expected with the extrudate being slightly staticky. The static of the extrudate did not impact the cutting process in any way.

The DR-0638 batch tested extrusion of Vitael® DLG 7505 A at 20% and 40% drug load. Vitael® DLG 7505 A is an acid terminated biodegradable PLGA copolymer. Its L/G ratio is 75:25, with IV range of 0.4-0.6. The mixing of VitaelR DLG 7505 A with ketamine hydrochloride at both loadings (e.g., 20% and 40% drug load) went as expected. The resulting blends visually looked homogeneous. The extrusion parameters were determined based on the melting temperature of Vitael DLG 7505 A and the observations from the trials in DR-0637, with temperature set at 150° C. and screw speed set at 25 rpm. The motor loads were 0.12 Nm and 0.19 Nm respectively for the two extrusions of 20% drug load and 0.28 Nm for 40% drug load. The conveyor belt was set at 1-10 fpm, while the actual values were 8 and 8.5 respectively for the two extrusions of 20% drug load and 8 for the 40% drug load. The temperature was increased to soften the surface finish of the implants. The tradeoff of the increased temperature resulted in increased throughput. Consequently, the diameter control was not as tight as possible. Even at the higher temperature, the resulting extrudate was visually white. Although there was some slight improvement on surface finish, the surface finish still looked rough overall (FIG. 2). From visual inspection, it was determined that at 150° C., there was no degradation of the formulations due to processing.

The DR-0639 batch tested extrusion of Vitael® DLG 7505 E at 20% and 40% drug load. Vitael® DLG 7505 A is an ester terminated biodegradable PLGA copolymer. Its L/G ratio is 75:25, with IV range of 0.4-0.6. The mixing of Vitael® DLG 7505 E with ketamine hydrochloride at both loadings (e.g., 20% and 40% drug load) went as expected. The resulting blends visually looked homogeneous. The extrusion parameters were determined based on the melting temperature of Vitael® DLG 7505 E and the observations from the trials in DR-0638, with temperature set at 140° C. and screw speed set at 25 rpm. The motor loads were 0.34 Nm and 0.36 Nm respectively for the two extrusions of 20% drug load and 0.75 Nm and 0.51 Nm respectively for the two extrusions of 40% drug load. The conveyor belt was set at 1-10 fpm, while the actual values were 8 for the 20% drug load and 8.5 for the 40% drug load. Overall, this set of conditions provided the tightest diameter control. The resulting extrudate looked visually white and the surface finish was rough (FIG. 3). From visual inspection, it was determined that at 140° C., there was no degradation of the formulations due to processing.

### In-Process Dimensional Analysis

The in-process dimensional analysis was performed on random extrudate stranding for each of the six formulations. 30 implants were cut to size for each formulation. Each implant was measured with a handheld micrometer for diameter (D) and length (L). The raw data was analyzed in terms of the maximum and minimum values as well as the average of all values. From the results, it was observed that DR-0639 resulted in the tightest diameter at both 20% and 40% drug load. Since there were no significant processing differences observed between runs, the difference in diameter measurements can be attributed to fact that the formulations were manually fed in the feed throat of the extruder. Fluctuations on diameter can be mitigated in the future by scale up. The constant feed rate of the T-12 feeder along with the metering of the 18-mm twin screw extruder and the draw-down of the puller/cutter can result in tighter diameter measurements.

Table 1 below provides a static analysis of ketamine HCl implants. The dimension of each individual implant is provided in Table 2 below.

TABLE 1
Static analysis of in-process dimension results

| | | DR-0637 | | | DR-0638 | | | DR-0639 | | |
|---|---|---|---|---|---|---|---|---|---|---|
| | | Max | Min | Average | Max | Min | Average | Max | Min | Average |
| 20% | Diameter, mm | 1.31 | 0.60 | 1.02 | 1.33 | 1.11 | 1.22 | 1.33 | 1.07 | 1.19 |
| | Length, mm | 29.1 | 14.0 | 22.6 | 22.62 | 16 | 20.1 | 21.83 | 19.35 | 20.4 |
| 40% | Diameter, mm | 1.28 | 0.92 | 1.14 | 1.41 | 1.15 | 1.29 | 1.34 | 0.88 | 1.21 |
| | Length, mm | 22.5 | 13.7 | 17.1 | 20.7 | 16 | 19.5 | 21.94 | 18.22 | 20.7 |

TABLE 2
Dimensions of exemplary ketamine HCl implants

| DR-0637 | | | | DR-0638 | | | | DR-0639 | | | |
|---|---|---|---|---|---|---|---|---|---|---|---|
| Formulation 20% | | Formulation 40% | | Formulation 20% | | Formulation 40% | | Formulation 20% | | Formulation 40% | |
| D, mm | L, mm | D, mm | L, mm | D, mm | L, mm | D, mm | L, mm | D, mm | L, mm | D, mm | L, mm |
| 0.84 | 19.5 | 1.10 | 17.0 | 1.26 | 19.8 | 1.25 | 20.0 | 1.23 | 20.0 | 1.21 | 20.2 |
| 1.17 | 29.1 | 1.10 | 16.8 | 1.25 | 22.6 | 1.33 | 16.0 | 1.21 | 19.7 | 1.21 | 20.2 |
| 0.82 | 27.9 | 0.92 | 16.8 | 1.15 | 20.2 | 1.30 | 19.8 | 1.23 | 19.7 | 1.31 | 20.9 |
| 0.88 | 19.1 | 1.18 | 18.8 | 1.24 | 21.0 | 1.30 | 20.1 | 1.21 | 20.3 | 1.30 | 20.2 |
| 0.93 | 17.5 | 0.99 | 20.1 | 1.16 | 19.3 | 1.28 | 20.0 | 1.24 | 19.7 | 1.26 | 20.5 |
| 0.60 | 19.5 | 1.14 | 15.0 | 1.11 | 20.9 | 1.29 | 20.2 | 1.27 | 19.8 | 1.26 | 20.5 |
| 0.96 | 14.0 | 1.18 | 173. | 1.33 | 20.0 | 1.35 | 18.9 | 1.27 | 20.2 | 0.88 | 18.2 |
| 0.91 | 26.8 | 1.13 | 14.3 | 1.17 | 20.8 | 1.29 | 20.3 | 1.27 | 20.2 | 1.34 | 21.0 |
| 1.0 | 23.6 | 1.15 | 18.8 | 1.28 | 20.3 | 1.28 | 20.3 | 1.33 | 20.0 | 1.13 | 21.1 |
| 1.23 | 22.0 | 1.15 | 19.9 | 1.22 | 17.6 | 1.21 | 19.2 | 1.21 | 20.4 | 1.27 | 20.6 |
| 1.31 | 27.2 | 1.09 | 13.7 | 1.24 | 21.2 | 1.29 | 19.8 | 1.23 | 20.5 | 1.18 | 20.3 |
| 0.84 | 27.3 | 1.02 | 18.0 | 1.27 | 19.3 | 1.34 | 18.3 | 1.15 | 20.5 | 1.11 | 20.0 |
| 1.0 | 25.2 | 1.17 | 15.0 | 1.30 | 18.5 | 1.30 | 19.3 | 1.13 | 20.3 | 1.29 | 21.9 |
| 1.02 | 21.5 | 1.02 | 19.9 | 1.23 | 19.7 | 1.41 | 20.7 | 1.19 | 20.1 | 1.20 | 21.7 |
| 1.20 | 22.9 | 1.21 | 19.6 | 1.23 | 22.4 | 1.35 | 19.6 | 1.10 | 21.3 | 1.23 | 21.0 |
| 0.89 | 19.4 | 1.22 | 17.0 | 1.19 | 19.7 | 1.18 | 16.0 | 1.23 | 20.8 | 1.22 | 20.4 |
| 0.99 | 19.4 | 1.15 | 17.1 | 1.29 | 20.7 | 1.15 | 18.5 | 1.16 | 20.6 | 1.29 | 19.7 |
| 0.91 | 27.7 | 1.22 | 18.8 | 1.29 | 20.9 | 1.27 | 19.4 | 1.20 | 20.6 | 1.21 | 21.2 |
| 0.89 | 17.4 | 1.23 | 17.4 | 1.17 | 21.1 | 1.40 | 19.5 | 1.13 | 20.1 | 1.10 | 21.7 |
| 1.12 | 20.3 | 1.24 | 14.4 | 1.17 | 18.6 | 1.31 | 20.6 | 1.15 | 21.5 | 1.10 | 21.0 |
| 1.05 | 20.5 | 1.26 | 14.3 | 1.17 | 19.7 | 1.18 | 19.5 | 1.13 | 20.9 | 1.30 | 21.4 |
| 1.04 | 28.1 | 1.12 | 17.8 | 1.29 | 21.1 | 1.22 | 19.2 | 1.18 | 21.8 | 1.18 | 20.9 |
| 0.98 | 27.1 | 1.28 | 15.0 | 1.24 | 19.1 | 1.31 | 19.7 | 1.14 | 21.4 | 1.27 | 20.8 |
| 1.16 | 20.5 | 1.14 | 17.6 | 1.28 | 19.0 | 1.28 | 19.9 | 1.11 | 21.7 | 1.06 | 20.8 |
| 1.20 | 19.2 | 1.06 | 15.0 | 1.19 | 20.5 | 1.19 | 20.5 | 1.33 | 19.6 | 1.24 | 21.6 |
| 1.15 | 26.1 | 1.21 | 14.7 | 1.21 | 20.6 | 1.25 | 19.4 | 1.21 | 20.1 | 1.27 | 20.7 |
| 1.16 | 19.7 | 0.97 | 16.1 | 1.28 | 19.5 | 1.32 | 19.3 | 1.17 | 19.4 | 1.18 | 21.9 |
| 1.14 | 23.7 | 1.20 | 17.6 | 1.12 | 16.0 | 1.26 | 20.6 | 1.14 | 19.9 | 1.25 | 21.1 |
| 1.17 | 23.7 | 1.09 | 16.4 | 1.14 | 21.1 | 1.34 | 20.4 | 1.07 | 20.6 | 1.11 | 19.9 |
| 1.06 | 22.3 | 1.18 | 22.5 | 1.15 | 20.5 | 1.33 | 19.3 | 1.19 | 19.7 | 1.27 | 20.5 |

### Drug Release Dissolution

Drug release dissolution can be tested via liquid chromatography on days 1, 3 and 7 for all 6 formulations with 3 pulls each and additionally on days 10, 14, 21 and 30 for two selected formulations for ongoing drug release testing with 4 pulls.

### Storage Stability

Storage stability testing can be conducted at 40° C. at 75% relative humidity (RH), with pull at 1 M on two selected formulations. The testing can last for 30 days.

### Example 2

### Treatment of Fibromyalgia using PLGA/Ketamine Implants

This Example describes the use of implants comprising ketamine hydrochloride disclosed herein for the treatment of fibromyalgia. The animals implanted with the ketamine-containing implants are inspected for symptoms of fibromyalgia periodically (e.g., daily, weekly, bi-weekly, monthly, every two months, every three months, every four months, every five months, every six months, or yearly). The methods disclosed herein improves the symptoms of fibromyalgia. Specifically, the methods disclosed herein are expected to reduce pain intensity, severity of tenderness, fatigue and stiffness and improves physical and mental functioning of the treated animals.

### Example 3

### Stability of PLGA/Ketamine Implants

This Example describes the drug release dissolution and storage stability of 2 different preclinical formulations of implants comprising ketamine hydrochloride for the treatment of fibromyalgia. The ketamine hydrochloride loaded implants had different bioresorbable polymers (e.g., Vitael® DLG 7505 A, or 7505 E).

The two batches tested in this Example were DR-0692 and DR-0693 extruded into cylindrical rods with Vitael® DLG 7505A and at DLG 7505E loaded with ketamine, respectively. Each batch has a batch size of 600 g. The storage stability is tested at: (1) 2-8° C.; or (2) 25° C. at 60% RH. Briefly, the implants (rods) were stored at either (1) 2-8° C.; or (2) 25° C. at 60% RH for a month before drug release dissolution was tested via liquid chromatography on Day 1, Day3, Day 7, Day 10, Day 14, Day 21 and Day 28.

In tables 3-6 below: (a) for development purposes, the cylindrical rods were off-white to beige, approximately 10.0 mm long×1.19 mm diameter, free from visible contamination, black spots, tails, jagged edges, irregular size and particulate matter (visually observed, n=10 implants); (b) related compounds refer to ICH Q3B (R2) impurities in drug product. Abbreviations ND=Not Detected and NR=Not Reported; (c) the date when in-house analytical laboratory testing completed; and (d) the date when outsourced (endotoxin) testing completed.

TABLE 3
stability tests of DR-0692 at 2-8° C.

| Test | | | Time Points (months) | | | |
|---|---|---|---|---|---|---|
| Method | Specification | | Release (0) | | 1 | |
| Appearance | N/A^{(a)} | | Conforms | | Conforms | |
| Inherent | N/A | | 0.45 dL/g | | NT | |
| Viscosity | | | | | | |
| Dissolution | N/A | | Avg. % Dissolution | | Avg. % Dissolution | |
| | | | Day 1 | 39.9% | Day 1 | 43.7% |
| | | | Day 3 | 71.9% | Day 3 | 71.3% |
| | | | Day 7 | 82.7% | Day 7 | 85.8% |
| | | | Day 10 | 84.7% | Day 10 | 78.0% |
| | | | Day 14 | 86.4% | Day 14 | 83.5% |
| | | | Day 21 | 90.5% | Day 21 | 85.6% |
| | | | Day 28 | 92.9% | Day 28 | 90.6% |
| Assay | N/A | | 99.5% | | 99.1% | |
| Related | Impurities | Limit | Impurities | Result | Impurities | Result |
| Compounds ^{(b)} | Related | N/A | Related | ND | Related | ND |
| | Compound A | | Compound A | | Compound A | |
| | Unspecified | | N/A | N/A | RRT 0.48 | NR |
| | Impurities | | N/A | N/A | RRT 0.52 | NR |
| | (Report all | | N/A | N/A | RRT 0.62 | NR |
| | individual | | RRT 0.76 | NR | RRT 0.76 | NR |
| | Unspecified | | RRT 0.84 | 0.32% | RRT 0.83 | 0.29% |
| | Impurities) | | RRT 0.95 | NR | N/A | N/A |
| | | | N/A | N/A | RRT 1.57 | NR |
| | | | RRT 1.89 | 0.13% | RRT 1.89 | 0.13% |
| | Total | N/A | Total | 0.45% | Total | 0.42% |

| DSC | N/A | 114.02° C. (Tg) | | 118.02° C. (Tg) |
|---|---|---|---|---|
| TGA | N/A | 1.11% | | 1.15% |
| Bacterial | N/A | Rep 1 | <0.0713 | NT |
| Endotoxins | | (1:100) | EU/Device | |
| USP<85> | | Rep 2 | <0.704 | |
| | | (1:1000) | EU/Device | |
| Sterility | No Growth | No Growth | | NT |
| USP<71> | | | | |

| Analysis | References | | |
|---|---|---|---|
| Information | Scheduled | N/A | 18 Nov. 2024 |
| | Pull Date | | |
| | Actual Pull Date | 17 Oct. 2024 | 18 Nov. 2024 |
| | Test Date Start | 8 Oct. 2024 | 19 Nov. 2024 |
| | Test Date | 6 Nov. 2024^{(c)} | 18 Dec. 2024 |
| | Completion | 10 Jan. 2025^{(d)} | |
| | Specification | N/A | N/A |
| | Revision | | |
| | WI-212 | 3.0 | 3.0 |
| | TM-25 | 0.1 | N/A |
| | TM-147 | 2.0 | 2.0 |
| | TM-145 | 3.0 | 4.0 |
| | TM-146 | 1.0 | 2.0 |
| | W1-191 | 7.0 | 7.0 |
| | WI-194 | 4.0 | 4.0 |
| | TR No. | TR-24-0348 | TR-24-0385 |

TABLE 4
stability tests of DR-0692 at 25° C.

| Test | | | Time Points (months) | | | |
|---|---|---|---|---|---|---|
| Method | Specification | | Release (0) | | 1 | |
| Appearance | N/A^{(a)} | | Conforms | | Conforms | |
| Inherent | N/A | | 0.45 dL/g | | NT | |
| Viscosity | | | | | | |
| Dissolution | N/A | | Avg. % Dissolution | | Avg. % Dissolution | |
| | | | Day 1 | 39.9% | Day 1 | 45.9% |
| | | | Day 3 | 71.9% | Day 3 | 73.5% |
| | | | Day 7 | 82.7% | Day 7 | 83.7% |
| | | | Day 10 | 84.7% | Day 10 | 77.6% |
| | | | Day 14 | 86.4% | Day 14 | 77.7% |
| | | | Day 21 | 90.5% | Day 21 | 87.0% |
| | | | Day 28 | 92.9% | Day 28 | 91.3% |
| Assay | N/A | | 99.5% | | 101.1% | |
| Related | Impurities | Limit | Impurities | Result | Impurities | Result |
| Compounds^{(b)} | Related | N/A | Related | ND | Related | ND |
| | Compound A | | Compound A | | Compound A | |
| | Unspecified | | N/A | N/A | RRT 0.48 | NR |
| | Impurities | | N/A | N/A | RRT 0.53 | NR |
| | (Report all | | N/A | N/A | RRT 0.62 | NR |
| | individual | | N/A | N/A | RRT 0.69 | NR |
| | Unspecified | | N/A | N/A | RRT 0.76 | 0.06% |
| | Impurities) | | RRT 0.76 | NR | RRT 0.83 | 0.24% |
| | | | RRT 0.84 | 0.32% | N/A | N/A |
| | | | RRT 0.95 | NR | RRT 1.57 | NR |
| | | | RRT 1.89 | 0.13% | RRT 1.90 | 0.12% |
| | Total | N/A | Total | 0.45% | Total | 0.42% |

| DSC | N/A | 114.02° C. (Tg) | | NT |
|---|---|---|---|---|
| TGA | N/A | 1.11% | | NT |
| Bacterial | N/A | Rep 1 | <0.0713 | NT |
| Endotoxins | | (1:100) | EU/Device | |
| USP<85> | | Rep 2 | <0.704 | |
| | | (1:1000) | EU/Device | |
| Sterility | N/A | No Growth | | NT |
| USP<71> | | | | |

| Analysis | References | | |
|---|---|---|---|
| Information | Scheduled Pull Date | N/A | 18 Nov. 2024 |
| | Actual Pull Date | 17 Oct. 2024 | 18 Nov. 2024 |
| | Test Date Start | 8 Oct. 2024 | 19 Nov. 2024 |
| | Test Date Completion | 6 Nov. 2024^{(c)} | 18 Dec. 2024 |
| | | 10 Jan. 2025^{(d)} | |
| | Specification | N/A | N/A |
| | Revision | | |
| | WI-212 | 3.0 | 3.0 |
| | TM-25 | 0.1 | N/A |
| | TM-147 | 2.0 | 2.0 |
| | TM-145 | 3.0 | 4.0 |
| | TM-146 | 1.0 | 2.0 |
| | W1-191 | 7.0 | N/A |
| | WI-194 | 4.0 | N/A |
| | TR No. | TR-24-0348 | TR-24-0386 |

TABLE 5
stability tests of DR-0693 at 2-8° C.

| Test | | | Time Points (months) | | | |
|---|---|---|---|---|---|---|
| Method | Specification | | Release (0) | | 1 | |
| Appearance | N/A^{(a)} | | Conforms | | Conforms | |
| Inherent | N/A | | 0.46 dL/g | | NT | |
| Viscosity | | | | | | |
| Dissolution | N/A | | Avg. % Dissolution | | Avg. % Dissolution | |
| | | | Day 1 | 42.3% | Day 1 | 40.6% |
| | | | Day 3 | 74.0% | Day 3 | 69.3% |
| | | | Day 7 | 82.3% | Day 7 | 85.0% |
| | | | Day 10 | 84.1% | Day 10 | 83.4% |
| | | | Day 14 | 85.4% | Day 14 | 87.8% |
| | | | Day 21 | 88.9% | Day 21 | 88.2% |
| | | | Day 28 | 92.6% | Day 28 | 89.5% |
| Assay | N/A | | 100.2% | | 100.0% | |
| Related | Impurities | Limit | Impurities | Result | Impurities | Result |
| Compounds^{(b)} | Related | N/A | Related | ND | Related | ND |
| | Compound A | | Compound A | | Compound A | |
| | Unspecified | | N/A | N/A | RRT 0.48 | NR |
| | Impurities | | N/A | N/A | RRT 0.53 | NR |
| | (Report all | | N/A | N/A | RRT 0.62 | NR |
| | individual | | RRT 0.76 | NR | RRT 0.76 | NR |
| | Unspecified | | RRT 0.84 | 0.33% | RRT 0.83 | 0.22% |
| | Impurities) | | N/A | N/A | RRT 1.57 | NR |
| | | | RRT 0.95 | NR | N/A | N/A |
| | | | RRT 1.89 | 0.14% | RRT 1.89 | 0.12% |
| | Total | N/A | Total | 0.47% | Total | 0.34% |
| DSC | N/A | | 117.44° C. (Tg) | | 115.71° C. (Tg) | |
| TGA | N/A | | 1.14% | | 1.05% | |
| Bacterial | N/A | | Rep 1 | | <0.0683 | NT |
| Endotoxins | | | (1:100) | | EU/Device | |
| USP<85> | | | Rep 2 | | <0.683 | |
| | | | (1:1000) | | EU/Device | |
| Sterility | N/A | | No Growth | | NT | |
| USP<71> | | | | | | |

| Analysis | References | | |
|---|---|---|---|
| Information | Scheduled Pull Date | N/A | 18 Nov. 2024 |
| | Actual Pull Date | 17 Oct. 2024 | 18 Nov. 2024 |
| | Test Date Start | 8 Oct. 2024 | 19 Nov. 2024 |
| | Test Date | 6 Nov. 2024^{(c)} | 18 Dec. 2024 |
| | Completion | 10 Jan. 2025^{(d)} | |
| | Specification | N/A | N/A |
| | Revision | | |
| | WI-212 | 3.0 | 3.0 |
| | TM-25 | 0.1 | N/A |
| | TM-147 | 2.0 | 2.0 |
| | TM-145 | 3.0 | 4.0 |
| | TM-146 | 1.0 | 2.0 |
| | W1-191 | 7.0 | 7.0 |
| | WI-194 | 4.0 | 4.0 |
| | TR No. | TR-24-0349 | TR-24-0387 |

TABLE 6
stability tests of DR-0693 at 25° C.

| Test | | | Time Points (months) | | | |
|---|---|---|---|---|---|---|
| Method | Specification | | Release (0) | | 1 | |
| Appearance | N/A^{(a)} | | Conforms | | Conforms | |
| Inherent | N/A | | 0.46 dL/g | | NT | |
| Viscosity | | | | | | |
| Dissolution | N/A | | Avg. % Dissolution | | Avg. % Dissolution | |
| | | | Day 1 | 42.3% | Day 1 | 41.8% |
| | | | Day 3 | 74.0% | Day 3 | 72.8% |
| | | | Day 7 | 82.3% | Day 7 | 86.7% |
| | | | Day 10 | 84.1% | Day 10 | 83.4% |
| | | | Day 14 | 85.4% | Day 14 | 89.5% |
| | | | Day 21 | 88.9% | Day 21 | 91.2% |
| | | | Day 28 | 92.6% | Day 28 | 91.6% |
| Assay | N/A | | 100.2% | | 98.4% | |
| Related | Impurities | Limit | Impurities | Result | Impurities | Result |
| Compounds^{(b)} | Related | N/A | Related | ND | Related | ND |
| | Compound A | | Compound A | | Compound A | |
| | Unspecified | | N/A | N/A | RRT 0.48 | NR |
| | Impurities | | N/A | N/A | RRT 0.53 | NR |
| | (Report all | | N/A | N/A | RRT 0.62 | NR |
| | individual | | N/A | N/A | RRT 0.69 | NR |
| | Unspecified | | RRT 0.76 | NR | RRT 0.76 | 0.06% |
| | Impurities) | | RRT 0.84 | 0.33% | RRT 0.83 | 0.19% |
| | | | RRT 0.95 | NR | N/A | N/A |
| | | | N/A | N/A | RRT 1.57 | NR |
| | | | RRT 1.89 | 0.14% | RRT 1.90 | 0.14% |
| | Total | N/A | Total | 0.47% | Total | 0.38% |
| DSC | N/A | | 117.44° C. (Tg) | | NT | |
| TGA | N/A | | 1.14% | | NT | |
| Bacterial | N/A | | Rep 1 | | <0.0683 | NT |
| Endotoxins | | | (1:100) | | EU/Device | |
| USP<85> | | | Rep 2 | | <0.683 | |
| | | | (1:1000) | | EU/Device | |
| Sterility | No Growth | | No Growth | | NT | |
| USP<71> | | | | | | |

| Analysis | References | | |
|---|---|---|---|
| Information | Scheduled Pull Date | N/A | 18 Nov. 2024 |
| | Actual Pull Date | 17 Oct. 2024 | 18 Nov. 2024 |
| | Test Date Start | 8 Oct. 2024 | 19 Nov. 2024 |
| | Test Date | 6 Nov. 2024^{(c)} | 18 Dec. 2024 |
| | Completion | 10 Jan. 2025^{(d)} | |
| | Specification | N/A | N/A |
| | Revision | | |
| | WI-212 | 3.0 | 3.0 |
| | TM-25 | 0.1 | N/A |
| | TM-147 | 2.0 | 2.0 |
| | TM-145 | 3.0 | 4.0 |
| | TM-146 | 1.0 | 2.0 |
| | W1-191 | 7.0 | N/A |
| | WI-194 | 4.0 | N/A |
| | TR No. | TR-24-0349 | TR-24-0388 |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of treating fibromyalgia in a subject in need thereof, comprising administering to the subject an implant comprising (i) a composition comprising at least one selected from the group consisting of ketamine, norketamine and a pharmaceutically acceptable salt thereof, and (ii) a polymer.

2. The method of claim 1, wherein the implant comprises enantiomerically pure S-(+)-ketamine, enantiomerically pure R-(−)-ketamine, or a racemic mixture of S-(+)-ketamine and R-(−)-ketamine.

3. The method of claim 1, wherein the implant comprises ketamine hydrochloride.

4. The method of claim 1, wherein the polymer is acid-terminated or ester-terminated.

5. The method of claim 1, wherein the polymer is selected from the group consisting of poly(lactic-co-glycolic acid) (PLGA) copolymer, polyesteramide, polyanhydride, polyacetal, polycaprolactone, polycarbonate, and any combination thereof.

6. The method of claim 1, wherein the polymer is PLGA copolymer.

7. The method of claim 6, wherein the PLGA copolymer has a co-monomer ratio for lactide to glycolide (L/G) content of about 50:50 to about 85:15.

8. (canceled).

9. The method of claim 1, wherein the composition is present in an amount of 10%-90% of the mass of the implant.

10.-13. (canceled)

14. The method of claim 1, wherein the implant has a mass of about 0.01-1 g, optionally 0.2-0.8 g.

15. The method of claim 1, wherein the implant is biodegradable.

16. The method of claim 1, wherein the implant exhibits a reduced pH internal environment.

17. The method of claim 16, wherein the reduced pH internal environment facilitates the release of the composition with enhanced solubility at reduced pH.

18. The method of claim 1, wherein the composition is released from the polymer over an extended period of time.

19. The method of claim 18, wherein the extended period of time is at least 14 days.

20. (canceled)

21. The method of claim 1, wherein the subject has a reduced pain intensity score, decreased tenderness, increased muscle endurance, decreased pain threshold and/or decreased pain tolerance.

22. The method of claim 21, wherein the pain intensity score is reduced by at least 20%, wherein the pain intensity score is measured by visual analog scale (VAS).

23. (canceled)

24. The method of claim 21, wherein the tenderness, pain threshold and/or pain tolerance is decreased by at least 20%.

25. The method of claim 21, wherein the muscle endurance is increased by at least 20%.

26. The method of claim 1, further comprising concurrently administering to the subject a muscle relaxant, an anti-inflammatory agent, or an antidepressant.

27.-31. (canceled)

32. The method of claim 1, wherein the administration is via a subcutaneous injection.
